(19) United States
(12) Patent Application Publication
(10) Pub. No.: US 2015/0056692 A1
(43) Pub. Date: Feb. 26, 2015

(54) Spouted Bed Bio-Reactor System

(71) Applicant: United Arab Emirates University, Al-Ain (AE)

(72) Inventors: Muftah H. El-Naas, Al-Ain (AE); Janice Arriesgado Acio, Al-Ain (AE); Ayat El Telib El Tayeb Hassan, Al-Ain (AE)

(73) Assignee: UNITED ARAB EMIRATES UNIVERSITY, Al-Ain (AE)

(21) Appl. No.: 13/975,834

(22) Filed: Aug. 26, 2013

(51) Int. Cl. C12M 1/12; C12M 1/02

(52) U.S. Cl. CPC C12M 25/20; C12M 41/22
USPC 435/303.1; 435/289.1

(57) ABSTRACT

The invention describes a novel spouted bed bioreactor system (SBBS) for the removal of Benzene, Toluene, Ethyl-benzene, o-,m-,p-Xylene (BTEX) from a contaminated air stream. Organic-degrading bacteria, Pseudomonas Putida, were immobilized in Polyvinyl Alcohol (PVA) matrices and utilized to degrade the BTEX in a specially-designed bioreactor system. The performance of the reactor system for the continuous biodegradation of BTEX in a contaminated air stream at different conditions was optimized.

### FIELD OF THE INVENTION

The invention provides a new environmentally friendly approach to the treatment of benzene, toluene, ethylbenzene, and xylene (BTEX). Specific strains of organics-degrading bacteria are immobilized in PVA matrices and utilized to degrade mixtures of BTEX compounds in a specially-designed Spouted Bed Bio-Reactor System (SBBS).

### BACKGROUND OF THE INVENTION

Benzene, toluene, ethylbenzene, and xylene (BTEX) are some of the most common air pollutants and have been subjected to increasingly stringent environmental regulations over the last two decades. Air emissions from different chemical, petrochemical and petroleum industries contain high concentrations of BTEX, which may have detrimental effects on public health and welfare. These compounds are naturally occurring constituents of gasoline, diesel and other petroleum products. They can be emitted during various oil and gas processing activities, including flaring, venting as well as dehydration and sweetening of natural gas. BTEX are suspected carcinogens and may lead to numerous health problems including leukemia through prolonged exposure.

A number of air pollution control (APC) technologies have been used for the removal of BTEX compounds from air steams. Adsorption, absorption, thermal oxidation, catalytic oxidation, and chemical scrubbing are the most common viable options for the treatment of volatile organic compounds including BTEX. However, applications of these conventional APC technologies have drawbacks in treatment of industrial BTEX emissions. A variety of methods have been developed to remove BTEX, including bio-filtration, incineration, stripping, membrane fractionation, scrubbing and physical adsorption (J. of Environ. Manag. 95 (2012), S55-S60).

Adsorption of VOCs on packed beds of solid adsorbents is an important and common method that has seen a lot of development in recent years, including the use of different adsorbents and different adsorption techniques (Int. J. Refrig., 29 (2006)22-29.; Anal. Chem. 73 (2001) 3449-3457). One of these techniques commonly used for the removal of BTEX is activated carbon adsorption (Physicochem. Eng. Aspects 214 (2003) 181-193.) Adsorption by activated carbon is considered an efficient technology for removing VOCs from air pollution sources, since they have large surface area, high porosity and rapid adsorption capabilities (for a review see: Renew. Sustain. Ener. Rev., 11 (2007) 1966-2005). However, the cost associated with highly expensive adsorbents such as activated carbon and the regeneration process makes adsorption a less desirable approach for industrial applications.

Another technique used for the removal of BTEX compounds is thermal oxidation. U.S. Pat. No. 6,964,729 describes an improved system for oxidizing undesired compounds residing within a liquid glycol based absorbent. The compounds are heated inside a re-boiler chamber until it reaches boiling point to maximize the production of vaporized effluents. The absorbent's vaporized effluents are then raised upwardly exiting the re-boiler chamber and going through a reflux tower, wherein they are partially condensed via a condenser embodied in the interior of the tower. The residual uncondensed effluents are then sent to be heated via a vaporizer, increasing the vaporization of any ambient condensed liquids contained within the effluents. The re-vaporized effluents then go into the system's thermal oxidizer combustion chamber where they are reheated to a temperature to effectuate elimination of undesirable compounds.

Although thermal oxidation is the most effective of these methods, it suffers from many drawbacks, including high energy requirements and the release of large amounts of CO₂ due to the use of fossil fuels. Studies that evaluated Global Warming Potential for VOCs showed that the use of fossil fuels in the incineration of VOCs may have a more negative climate change effects than the release of these compounds into the air (AEA Ener. & Environ. 3 (2007), 4-26).

Sweetening has also been used in removing residual VOC components from a fluid stream. U.S. Pat. No. 6,605,138 describes a technique for removing “VOC” (Volatile Organic Compounds) starting by removing station from primary VOC in an absorbent fluid stream with interposing supplemental VOC removal station. The VOC and sour gas components are absorbed by the absorbent fluid stream, while the supplemental removal station is used to liberate a portion of the residual absorbed VOC left over in the absorbent fluid stream, which is downstream from the removal station.

Compared with physical and chemical methods, biological treatment is becoming an increasingly popular technique for air pollution control. It often offers a cost-effective solution for the treatment of large volumetric airstreams containing low levels of pollutants (Biofiltration for Air Pollution, Devinny, J. S., Deshusses. 1999). It is an alternative to conventional air pollution control technologies such as thermal or catalytic oxidation, wet scrubbing, and adsorption onto activated carbon. Biological treatment is achieved at ambient temperatures and does not generate secondary pollutants; it converts volatile organic compounds to carbon dioxide, reduces sulfur compounds to sulfate, and chlorinated compounds to CO₂ and chloride (Curr. Opin. Biotechnol. 9 (1998) 256-262). Several bioreactors have been developed for treating volatile organic compounds and odorous compounds in vapor phase. The different types of air phase biological reactors include biofilters, biotrickling filters and bioscrubbers (J. Environ. Manag 91 (2010), 1039-1054). Among the newly developed reactors are the membrane reactors (Chem. Eng. J. 136 (2008), 82-91); novel rotating rope bioreactor (Bioresource Technol. 99 (2008) 1044-1051); bioactive foam emulsion reactor (Chinese J. of Chem. Eng 18 (2010), 113-121); a flat plate vapor phase bioreactor using oxygen micro sensors (Water Sci. Techn. 36 (1997), 77-84); and two liquid phases bioreactor (J. Hazard. Mater. 148 (2007), 453-458). Some other examples are the external loop airlift bioreactor (J. of Chem. Techno. & Biotechnol. 78 (2003), 406-411); fluidized bed bioreactors (Interna, J. of Chem. Reactor Eng. 5 (2007), A22); and monolith bioreactor (Proc. Biochemis. 43 (2008), 925-931). Biodegradation has attracted the attention of many researchers as an effective technique for the removal of BTEX from contaminated air (J. of Chem. Techno. and Biotechnol. 72 (1998)303-319), water (Environ. Pollu. 107 (2000), 187-197) and soil (J. Hazard. Mater. 46 (1996), 1-12).

Compared to the biodegradation of a single contaminant, the biodegradation of BTEX mixture has not been well-studied and rarely reported in the literature. Only limited studies have been reported on the biodegradation of mixtures (Proc. Biochemis. 40 (2005) 2015-2020); (Bioresou. Technol. 99 (2008), 7807-7815); (Chem. Eng. J. 172 (2011), 735-745); (Proc. Biochemis. 39 (2004), 983-988).

US2012/0142083 describes a biofilter used for treating contaminated gases with a layer of rubber particulate, which is a filter media. The source of the rubber particulate is recycled tires. The rubber particulate provides a platform for the growth and maintenance of a microbial ecosystem that substantially treats the contaminated gases. Also, the invention claims to reduce odorous pollutants from products wherein the product, containing the pollutant or pollutants, is passed through a layer of the rubber particulate material.

The main advantage of bio-filters is the conversion of BTEX compounds into harmless oxidation products, but their performance relies on constant loading (Worl., J. of Microbiol. and Biotechno, 21 (2005) 787-789). In fact, most bio-filters require a pretreatment step that may involve adsorption (Bacterial channels and their eukaryotic homologues, A. Kubalski, and B. Martinac, 247-258) or surfactant-aided absorption to reduce and stabilize the concentration of the BTEX before the biotreatment (Gener. of huma-induced pluripotent stem cells. Nat. Protocols 3 (2008), 1180-1186).

US2012/0227587 provides a system for venting and filtration using a membrane, which is permeable to gas and substantially impermeable to liquid. The systems can remove a gas from a liquid entrained with gas. The systems can contain a reservoir in fluid communication with the membrane and a liquid outlet. In addition, U.S. Pat. No. 5,985,649 describes an invention for the removal of gas contaminants by dispersing the contaminated gas though a fluid filled container having free biomass suspended, where the biomass is selected to metabolize the gas contaminants requiring degradation. This system, however, could suffer from biofouling, channeling and biomass carry over. In general, bioreactors have difficulties in long term operation such as fouling formation and difficult scale-up, which limit their practical application in the field in spite of their high mass transfer and biodegradation rates.

### SUMMARY OF THE INVENTION

In a first aspect, the present invention provides a system for removing contaminants from a stream of fluid, comprising at least one spouted bed reactor, the spouted bed reactor comprising:
i. a vertically extending cylinder with a fluid inlet orifice at the bottom and a fluid outlet orifice at the top;
ii. nutrient media; and
iii. immobilised bacteria,
   wherein the immobilised bacteria are capable of degrading the contaminants.

Preferably, the vertically extending cylinder has a conical or frusto-conical base, the fluid inlet orifice being at the apex of the cone. In certain preferred embodiments, the system comprises two or three spouted bed reactors in series.

Preferably, the bacteria are immobilised in polyvinyl alcohol. Preferably, the bacteria comprise bacteria from the Pseudomonas genus. More preferably, the bacteria comprise Pseudomonas putida.

In a preferred embodiment, the nutrient media is essential mineral media comprising magnesium sulphate, dipotassium hydrogen phosphate, calcium chloride, ammonium carbonate, ferrous sulphate, zinc sulphate, manganese(II) dichloride, copper sulfate, cobalt dichloride and sodium molybdate.

Most preferably, the contaminants to be removed are benzene, toluene, ethylbenzene, and xylene (BTEX).

In a preferred embodiment, the flow rate of fluid through the reactor or reactors is around 100, 200, 300, 400, 500, 600, 700, 800, 900, 1000, 1250, 1500, 1750, 2000, 2250 or 2500 ml per minute. Most preferably, the flow rate is around 500 ml per minute.

In another preferred embodiment, the temperature of the reactor or reactors is controlled with a water jacket or jackets. In a further preferred embodiment, the temperature in the reactor or reactors is held at around 25, 30, 35 or 40° C. Most preferably, the temperature in the reactor or reactors is held at around 35° C.

In yet another preferred embodiment, the pH of the nutrient media is around 5, 6, 7, 8 or 9. Most preferably, the pH of the nutrient media is around 8.

### BRIEF DESCRIPTION OF THE DRAWINGS

FIG. 1: A flow diagram of the spouted bed bioreactor system.

FIG. 2: A schematic diagram of the spouted bed bioreactor.

FIG. 3: BTEX removal for different flow rates

FIG. 4: BTEX removal for different operating temperatures

FIG. 5: BTEX removal for different PVA volumes

FIG. 6: BTEX removal for different values of solution pH

### DETAILED DESCRIPTION OF THE INVENTION

In this invention, a more efficient biodegradation process is presented. Specific strains of organics-degrading bacteria are immobilized in PVA matrices and utilized to degrade mixtures of BTEX compounds in a specially-designed three Spouted Bed Bioreactors in series. The aim is to utilize the main advantages of biodegradation and eliminate the drawbacks associated with bio-filters through the use of well mixed moving bed of bacteria immobilized in PVA gel pellets. Besides the use of a novel bioreactor system, mixtures of BTEX compounds are degraded simultaneously or sequentially in the same bioreactor.

The term “spouted bed reactor” refers to a particular form of fluidised bed reactor. The term “spouted bed bioreactor” is used herein interchangeably. In a spouted bed reactor a fluid stream is injected at the base of a cylinder, which is filled with another fluid or powder. The cylinder may be constructed from any suitable material, including but not limited to metals, plastics, glasses and ceramics. Typically, the base of a spouted bed reactor will be in the form of a cone or conical frustum, with the fluid inlet being at the apex of this structure. This fluid inlet is sometimes referred to as the spouting orifice. Spouted bed reactors have the advantage of providing excellent mixing, particularly where the reactor contains large or irregularly sized particles.

It will be understood by those of skill in the art that the term “fluid” encompasses both liquids and gases. In preferred embodiments of the present invention the injected stream is a gas, for example contaminated air. A reactor in which a gas stream is passed through a liquid containing solid particles is sometimes referred to as a three phase reactor.

The present inventors have found that removal rates can be improved via the use of multiple reactors (either in series or in parallel) versus a single reactor of equal volume. Without wishing to be bound by a single theory, the inventors attribute this improvement to improved mixing leading to higher availability of the contaminants to the immobilised bacteria.

In preferred embodiments, the system comprises three reactors connected in series. It will be clear to those of skill in the art that “in series” is intended to mean that the outlet from the first reactor is connected to the inlet of the second reactor and that the outlet of the second reactor is connected to the inlet of the third reactor. This is in contrast to a parallel reactor configuration, in which multiple reactors would share a single inlet and outlet.

The flow rate of fluid through the reactor or reactors can be adjusted to achieve optimum contaminant removal. In a preferred embodiment, the flow rate is around 100, 200, 300, 400, 500, 600, 700, 800, 900, 1000, 1250, 1500, 1750, 2000, 2250 or 2500 ml per minute. Most preferably, the flow rate is around 500 ml per minute. Similarly, the temperature within the reactor or reactors can be controlled to achieve optimum contaminant removal. In a preferred embodiment temperature control is achieved via the use of a water jacket around the reactor, through which water of the desired temperature may be passed. In a further preferred embodiment, the temperature in the reactor or reactors is held at around 25, 30, 35 or 40° C. Most preferably, the temperature in the reactor or reactors is held at around 35° C.

In addition to flow rate and temperature, the pH of the liquid medium can also be controlled to achieve optimum contaminant removal. This may be achieved by any suitable means, including but not limited to the use of buffers. In a preferred embodiment the pH is around 5, 6, 7, 8 or 9. Most preferably, the pH is around 8.

A number of methods may be employed to immobilise bacteria within the spouted bed reactor of the invention. Immobilization of the bacterial biomass is an important and valuable technique that is usually employed to serve several purposes, including protection of the bacteria from high feed concentrations as well as ease of separation and reutilization of the biomass. In a particularly preferred embodiment, bacteria are immobilised within polyvinyl alcohol gel, which is subsequently cut into cubes. The dimensions of the cubes may be varied according to application, but the preferred size is 10 mm.

Any bacteria capable of degrading contaminant chemicals may be used in the present invention. In a preferred embodiment, the bacteria comprise species from the Pseudomonas genus, preferably Pseudomonas putida. The bacteria may be obtained from any suitable source, including but not limited to isolation from Amnite Cereal.

Various forms of nutrient media may be used in the spouted bed reactor of the present invention. In a preferred embodiment, essential mineral media is used. An example of the composition of essential mineral media is as shown in Table 1:

TABLE 1
Example composition of essential mineral media

| Component | Concentration (gm⁻³) |
|---|---|
| MgSO₄•7H₂O | 300 |
| K₂HPO₄ | 250 |
| CaCl₂•2H2O | 150 |
| (NH₄)₂CO₃ | 120 |
| FeSO₄•7H₂O | 3.5 |
| ZnSO₄•7H₂O | 1.3 |
| MnCl₂•4H₂O | 0.13 |
| CuSO₄•5H₂O | 0.018 |
| CoCl₂•6H₂O | 0.015 |
| Na₂MoO₄•2H₂O | 0.013 |
| Total | 824.976 |

### Example

The biodegradation of BTEX with P. Putida immobilized in PVA gel in a novel spouted bed bioreactor system was evaluated at different conditions. Experimental data showed that the BTEX concentration in the outlet gas stream was considerably reduced due to the biodegradation capabilities of the immobilized bacteria. As the BTEX gas bubbles through the reactor system it dissolves in water and essential mineral nutrients, which makes it accessible for the bacteria to be consumed as a sole carbon source.

The following conclusions were drawn from the results presented in this study: (a) P. Putida showed a good ability to remove BTEX with the maximum removal efficiency of 95%, 97%, 97%, 96% for benzene, toluene, ethylbenzene, and m,p,o-Xylene, respectively under the conditions illustrated in this study; (b) longer residence time at low flow rate of 500 ml/min provided the highest BTEX biodegradation rate; (c) optimum degradation rates are obtained at a temperature of 35° C. and a pH of 8. The experimental results clearly confirmed the effectiveness of the SBBS as a new tool for the biodegradation of difficult hydrocarbons such as BTEX and gave hope for the development of an effective and economical technology for the removal of these harmful pollutants.

### Extraction of Bacterial Culture

Pseudomonas putida was extracted from Amnite Cereal (P300) obtained from Cleveland Biotech Ltd., UK. The Amnite product contains 10 strains belonging predominantly to the Pseudomonas genera (main strain is P. putida). The component strains include both aerobic and facultative organisms. The total viable count in the ready to use product is not less than 5.0×10⁸ cfu/g.

The bacteria were extracted as follows: 100 g of the cereal was mixed in a 1000 ml of distilled water with 0.22% Sodium Hexametaphosphate. The mixture was homogenized in a blender for about an hour while adjusting the pH to 8.5 by adding Na₂CO₃ then decanted and refrigerated for 24 hours. Bacterial suspension was centrifuged at 6000 rpm for 15 minutes. Supernatant was collected and centrifuged again at 10000 rpm for 20 minutes. The biomass attaching to the walls of tubes was suspended in a small amount of distilled water and used as mixed bacterial culture.

### Immobilization of the Biomass

Polyvinyl alcohol (PVA) gel was used for immobilizing the biomass. A homogenous 10 wt % PVA was prepared by mixing 50 g of PVA powder with 450 ml of distilled water at about 70-80° C. The mixture was cooled to room temperature before adding 10 ml of the previously prepared bacterial suspension and stirring for about 15 minutes to ensure homogeneity of the solution. The mixture was then poured into special molds and kept in a freezer at −20° C. for 24 hrs before transferring to the refrigerator and allowed to thaw at about 4° C. for 5 hours. The freezing-thawing process was repeated for four cycles. The frozen molds were then cut into equal 10 mm cubes, washed with distilled water to remove any uncrossed-linked chains, and then used for acclimatization, where the biomass was acclimated to high concentrations of toluene over a period of 5 days (J. Hazard. Mater. 164 (2009), 720-725). The immobilised biomass used in the experiments described in the present application was used over an extended period (over one year) and remained active. The data presented below were collected four months after immobilisation.

### Spouted Bed Bioreactor System (SBBS)

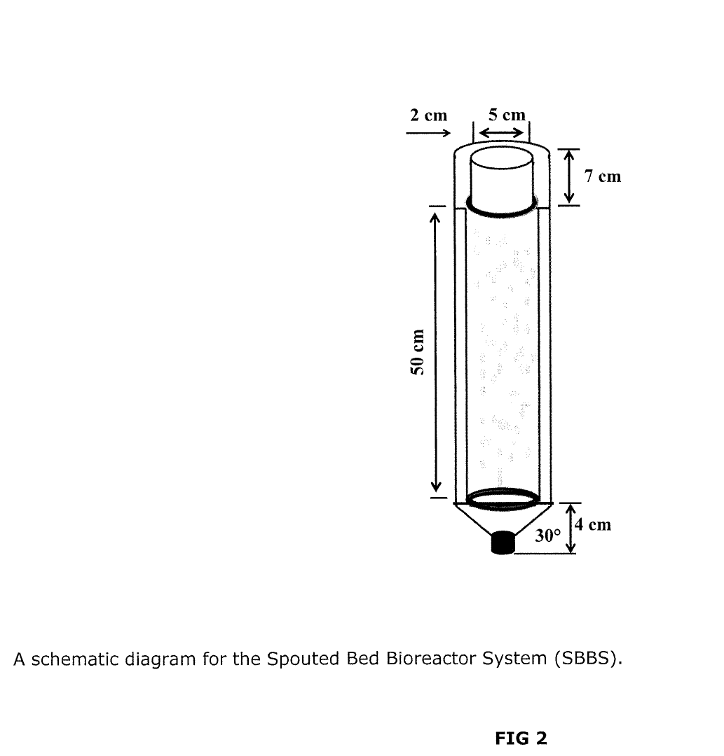

The SBBS was made of three Plexiglas columns (5 cm diameter, 57 cm length, frustoconical base with 4 cm height, 30° angle—see FIG. 2), each equipped with surrounding jackets for temperature control. The three one-liter reactors were placed in series and were initially filled with essential mineral media and PVA gel cubes containing immobilized bacteria. The temperature of the reactor system was controlled by circulating water into the reactor jacket from a water bath set at the desired value.

### Analytical Method

Samples were taken by an automatic sampling system at 7-minute time intervals by a Varian 3800 gas-liquid chromatograph (USA), equipped with a flame ionization detector. Separation was carried out on a CP-sil 8CB 30 m×0.32 mm ID, 1 μm film thickness fused silica capillary column using Helium as carrier, at a pressure of 18 psi. The column temperature was programmed at 40° C./min for 1 min, heating with 35° C./min rate to 90° C. held for 1 min and raising the temperature with 8° C./min to 114° C. Injector and detector temperatures were 200° C. and 250° C., respectively. Sierra Side-Trak™ and Auto-Trak™ mass flow meters and controller were used for accurately measuring and controlling gas flow rates (BTEX mixture Air). Standard output for the transducers is 0-5V DC signal which corresponds to 0 to 5000 ml/min mass flow.

### Experimental Setup

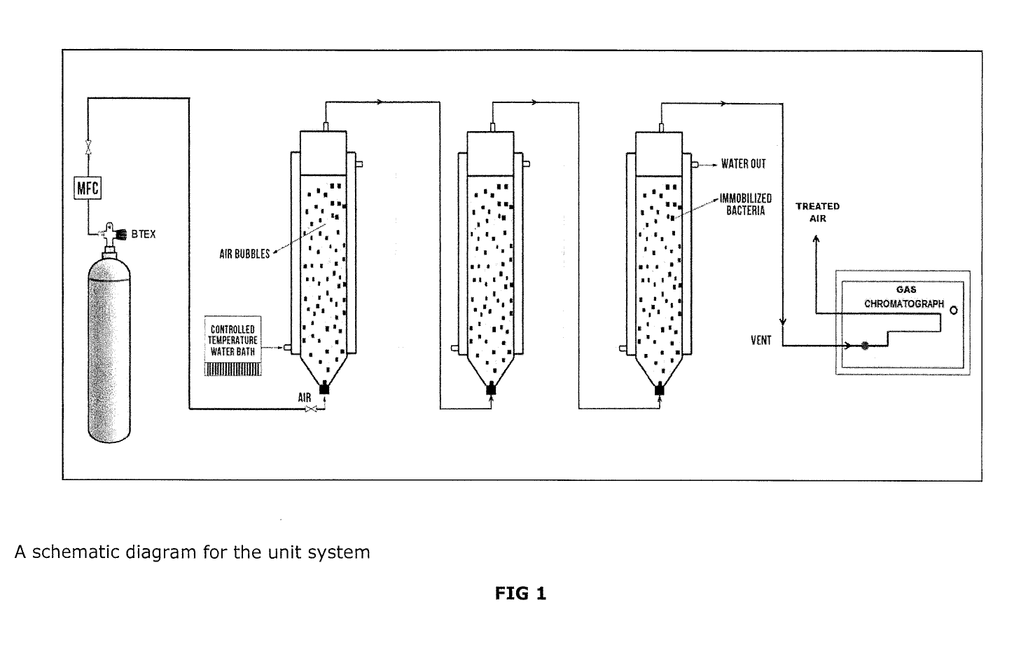

FIG. 1 shows a flow diagram of the spouted bed bioreactor system. Contaminated air is continuously introduced through the spouting orifice at the bottom of the reactor at constant flow rates which enhance mixing and at the same time providing oxygen and sustaining aerobic condition. The air going out from the top of the first is introduced again to the second reactor and the same goes to the third reactor. The dimensions of the spouted bed bioreactor are shown in FIG. 2. Each bioreactor contained PVA particles in nutrient medium mineral solution. The effluent air stream was continuously analyzed using gas chromatography to determine the efficiency of the degradation process. The rateS of BTEX removal in this set up are shown in Table 2:

TABLE 2
Rate of BTEX removal in a SBBS system. Three reactors
in series were used with a flow rate of 500 ml/min
at a temperature of 35° C. and a pH of 8.

| | Initial Concentration | Final Concentration | % Removal |
|---|---|---|---|
| Component | (ppm) | (ppm) | (1-X/X0) |
| Benzene | 10 | 0.46 | 95% |
| Toluene | 10 | 0.30 | 97% |
| Ethylbenzene | 10 | 0.32 | 97% |
| m-Xylene | 10 | 0.39 | 96% |
| p-Xylene | 10 | 0.39 | 96% |
| o-Xylene | 10 | 0.37 | 96% |

### Effect of Flow Rate

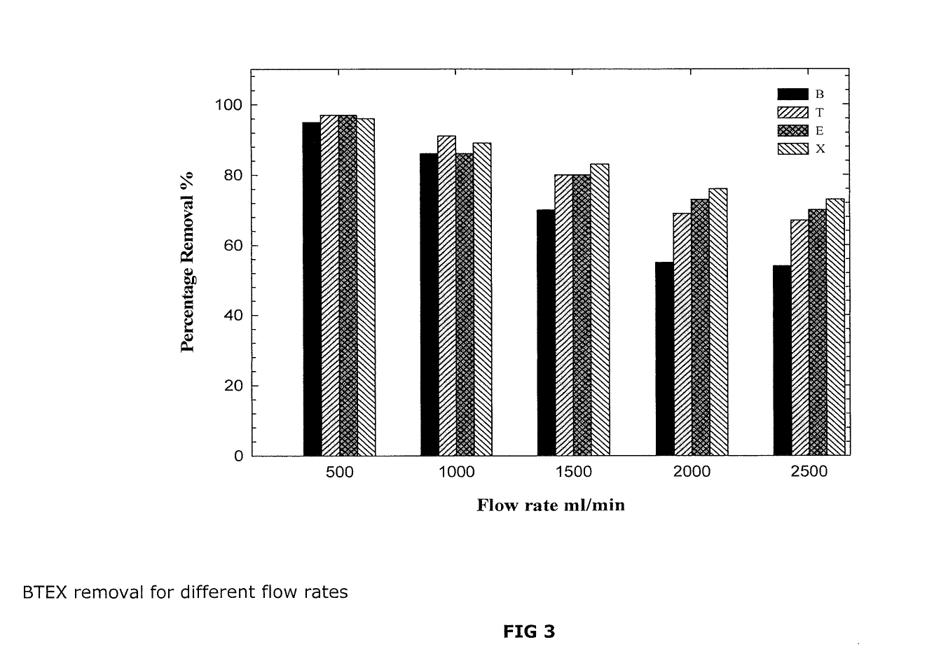

FIG. 3 presents the experimental results of the effect of flow rate on the % removal of BTEX. All parameters were kept constant; the total operating volume of the bioreactors was fixed at 3 liters; the PVA pellets represented 30% of the reactor volume, with the balance nutrient solution at 35° C.; while varying the flow rate from 500 to 2500 ml/min. It is noticeable that the reduction in BTEX concentration decreases with increasing flow rates. This can be attributed to the residence time of gas in the reactor. As expected, longer residence time results in higher biodegradation rate. The theory is that the increased solubility of the pollutants into the liquid phase will make them more bioavailable. By lowering airflow rates, the bacteria will have more contact time with the pollutants, and this will consequently increase the solubility and result in high degradation rates (G. T. Kleinheinz, B. A. Niemi, and J. T. Hose, “Proc. 92nd Annu. Meet. Air Waste Manag. Assoc.). Low flow rate (500 ml/min) provided the highest BTEX removal rate of 95% for benzene, 97% for toluene, 97% for Ethyl benzene and 96% for xylenes.

### Effect of Temperature

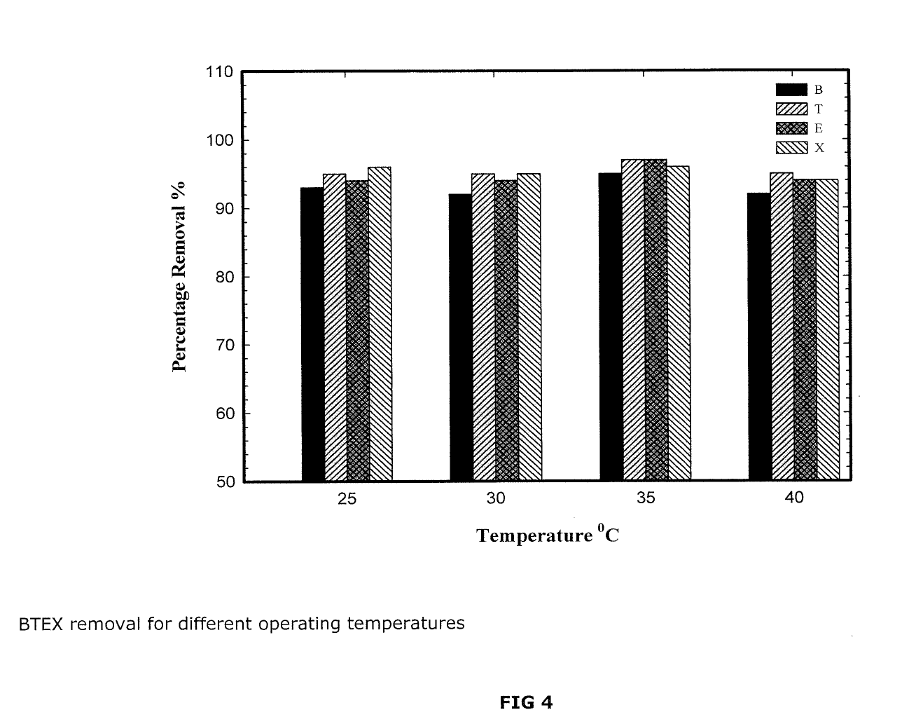

A plot of the BTEX removal percentage versus temperature is shown in FIG. 4. All parameters were kept constant, while varying the temperature from 25 to 40° C. Higher temperature has a negative effect on the bacteria and percentage removal. In fact, sudden exposure to temperature higher than 35° C. might have negative effect on bacterial enzymes that are usually responsible for benzene ring cleavage, which is the major step in the biodegradation process. In addition, low temperature is expected to slow down the bacteria activity. The optimum temperature for the maximum specific growth rate of Pseudomonas putida falls between 30 and 35° C. (Chemosphere, vol. 54 (2004), 1255-1265). This is clearly reflected in the biodegradation rate of BTEX, with 35° C. being the optimum temperature.

### Effect of PVA Volume

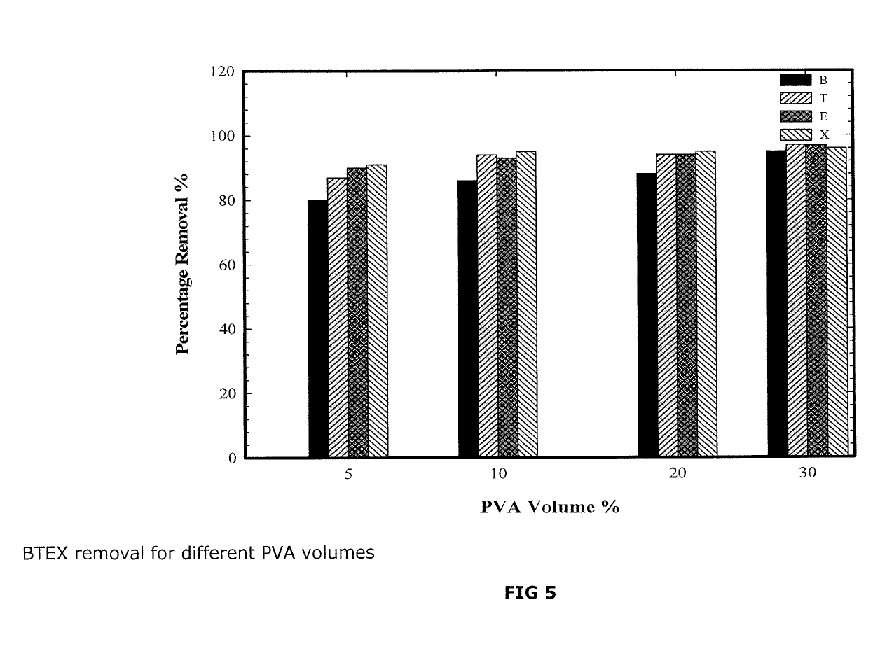

In this part of study, experiments were carried out to estimate the effect of PVA volume on % BTEX removal. The amount of PVA pellets which house the active biomass in the bioreactor plays a significant role in the removal. The initial BTEX concentration and temperature were kept constant 10 mg/l and 35° C., respectively. Also the total operating volume of the spouted bed bioreactors was kept constant at 3 liters with flow rate of 500 ml/min. The amount of PVA in the reactor is directly related to the amount of biomass; therefore, as the PVA volume increases the % BTEX removal increases. FIG. 5 shows PVA volumes ranging from 5 to 30%. As expected, the 30% PVA has the highest biodegradation rate because the reactor has more biomass.

Numerous effective degradation tests were carried out over more than one year and the bacteria is still active and working well. Furthermore, hydrocarbon removal without immobilized bacteria was tested using single component (toluene) in two reactors. The maximum rate of removal was about 15%, which can be attributed to toluene solubility in water. Once the water in the reactors was saturated, there was no further removal of toluene.

### Effect of Solution pH

Experiments were carried out to evaluate the effect of solution pH on the biodegradation of BTEX. All parameters were kept constant: operating temperature of 35° C.; the PVA pellets represented 30% of reactor volume; and BTEX contaminated air flow rate of 500 ml/min. The solution pH was varied from 6 to 9 using a few drops of HCl or NaOH depending on the desired value. The initial pH of the essential mineral media solution was about 8.

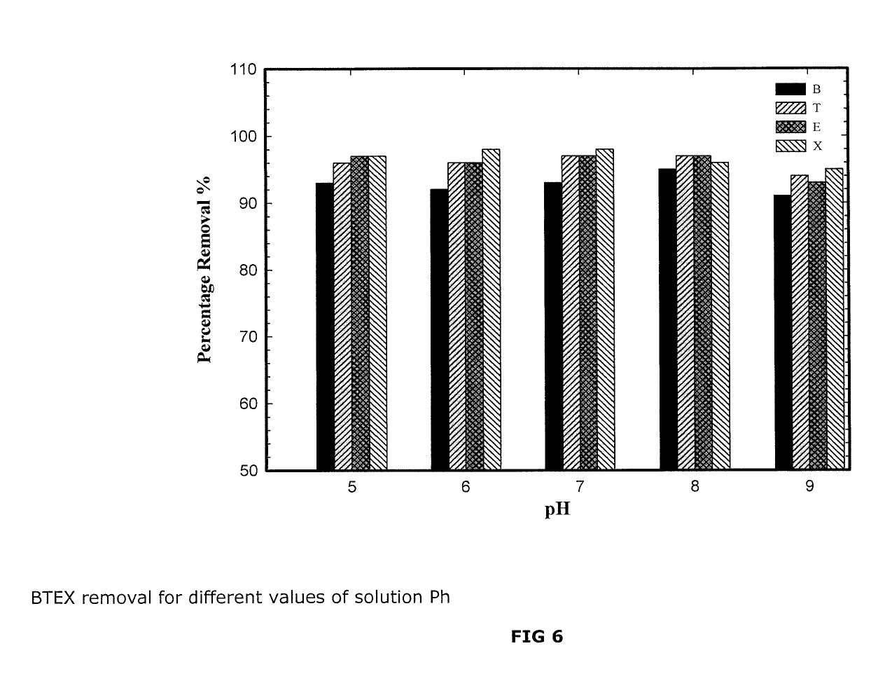

The experimental results, which are shown in FIG. 6, reveal that the removal percentage increases with pH reaching the maximum at a pH of 8. As is the case with several biological processes, pH has an important influence on bioreactor efficiency. The physiological pH effect on microbial activity is similar to the effect of temperature. All species have optimal pH conditions, some are more tolerant to a wider pH range, but some prefer a lower pH range (Afr. J. Biotechnol. 10 (2011), 13299-1330). The removal efficiency of BTEX was the maximum at 95% for benzene, 97% for ethyl benzene, and 96% for xylenes which clearly favored a weak basic environment at pH 8.

1. A system for removing contaminants from a stream of fluid, comprising at least one spouted bed reactor, the spouted bed reactor comprising:
i. a vertically extending cylinder with a fluid inlet orifice at the bottom and a fluid outlet orifice at the top;
ii. nutrient media; and
iii. immobilised bacteria,
wherein the immobilised bacteria are capable of degrading the contaminants.

2. The system of claim 1, wherein the vertically extending cylinder has a conical or frusto-conical base, the fluid inlet orifice being at the apex of the cone.

3. The system of claim 1, comprising two or three spouted bed reactors in series.

4. The system of claim 1, wherein the bacteria are immobilised in polyvinyl alcohol.

5. The system of claim 1, wherein the bacteria comprise bacteria from the Pseudomonas genus.

6. The system of claim 5, wherein the bacteria comprise Pseudomonas putida.

7. The system of claim 1, wherein the nutrient media is essential mineral media comprising magnesium sulphate, dipotassium hydrogen phosphate, calcium chloride, ammonium carbonate, ferrous sulphate, zinc sulphate, manganese(II) dichloride, copper sulfate, cobalt dichloride and sodium molybdate.

8. The system of claim 1, wherein the contaminants to be removed are benzene, toluene, ethylbenzene, and xylene (BTEX).

9. The system of claim 1, wherein the flow rate of fluid through the reactor or reactors is around 100, 200, 300, 400, 500, 600, 700, 800, 900, 1000, 1250, 1500, 1750, 2000, 2250 or 2500 ml per minute.

10. The system of claim 9, wherein the flow rate is around 500 ml per minute.

11. The system of claim 1, wherein the temperature of the reactor or reactors is controlled with a water jacket or water jackets.

12. The system of claim 1, wherein the temperature in the reactor or reactors is held at around 25, 30, 35, or 40° C.

13. The system of any claim 12, wherein the temperature in the reactor or reactors is held at around 35° C.

14. The system of claim 1, wherein the pH of the nutrient media is around 5, 6, 7, 8 or 9.

15. The system of claim 14, wherein the pH of the nutrient media is around 8.
